(19) United States
(12) Patent Application Publication
(10) Pub. No.: US 2011/0002444 A1
(43) Pub. Date: Jan. 6, 2011

(54) MODEL BASED SELF-POSITIONING PATIENT TABLE FOR X-RAY SYSTEMS

(75) Inventors: Holger Schmitt, Hamburg (DE); Udo Van Stevendaal, Ahrensburg (DE); Peter Forthmann, Sandesneben (DE); Michael Grass, Buchholz In Der Nordheide (DE)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/920,333

(22) PCT Filed: Mar. 9, 2009
(86) PCT No.: PCT/IB2009/050958

§ 371 Date: Aug. 31, 2010

(30) Foreign Application Priority Data
Mar. 12, 2008 (EP) 08102523.1

(51) Int. Cl. A61B 6/04; G01N 23/04

(52) U.S. Cl. 378/62; 378/205

(57) ABSTRACT

A System (24) for positioning a carrier (9) of an object (10) within a field of view of an imaging unit (7b), the system comprising: an imaging receiving interface unit (14) for receiving image data (22) representing an image (9) of the object (10) from an imaging unit (7b), an object components position determination unit (15) for determining object components (11, 12) position data (25) representing positions of components (11, 12) of the object (10), a memory (16) with anatomic model data stored therein, the anatomic model data representing anatomic model (9*) component positions of anatomic model components (11*, 12*) of an atomic model (10*), a matching unit (17) designed to match object components (11, 12) positions and anatomic model components (11*, 12*) positions based on object components position data and anatomic model data, an input receiving unit (19) for receiving input data (23) representing a selected component (11) of interest, a positioning planning unit (18) for determining position shift data representing a direction and a distance by which the carrier (8) is to be shifted, considering the input data, anatomic model data and object components position data, an interface (20) to a positioning system (21) for shifting the carrier (8) based on the position shift data (27).

### FIELD OF THE INVENTION

The invention concerns systems, detectors, methods and memory devices for positioning a carrier of an object within a field of view of an imaging apparatus,

### BACKGROUND OF THE INVENTION

EP 1 092 391 A1 discloses positioning a carrier of a human body within a field of view of an X-ray imaging apparatus depending on user input indicating amount and direction of a shift of the body after generating a first X-ray image of the human body.

### SUMMARY OF THE INVENTION

It is an object of the present invention to improve the prior art. This is accomplished by what is set forth in the appended independent claims, while the appended dependent claims define advantageous modifications thereof.

The invention allows efficient three-dimensional imaging on C-arm systems Before data for 3D reconstruction can be acquired, the examined organ, for example the heart, can efficiently be positioned in the iso-center of rotation of the C-arm such that it remains in the field of view during the entire rotational scan. The positioning according to the invention is efficient regarding time and X-ray dose.

Specifically, according to a first aspect of the present invention described in claim 1, there is provided a system for positioning a carrier of an object within a field of view of an imaging unit, the system comprising:
an image receiving interface unit for receiving image data, representing an image of the object, from the imaging unit,
an object components position determination unit for determining object components position data representing positions of components of the object,
an anatomic model data memory with anatomic model data stored therein, the anatomic model data representing anatomic model component positions of anatomic model components of an atomic model,
a matching unit designed to generate, based on object components position data and anatomic model data,
image data representing an image, which image data is to be provided via an interface to an image display unit,
an input receiving unit for receiving input data representing a selected component of interest,
a positioning planning unit for determining position shift data, depending on at least the input data, representing a direction and a distance by which the carrier is to be shifted,
an interface to a positioning system for shifting the carrier based on the position shift data.

Preferably the object is a human body and the components of the objects are at least one of: organs and bones of the human body.

Preferably the carrier is a table.

Preferably the image data represents only one image of a front view or rear view of the object.

Preferably object position data represents a three-dimensional position of an object.

Preferably it further comprises a display unit displaying an image representing anatomic model components positions and object components positions.

Preferably anatomic model components positions are average positions of the components within the object.

Preferably the imaging apparatus is a cardio-vascular X-ray system or other X-ray system.

Preferably the imaging apparatus comprises a rotatable C-arm.

According to a second aspect of the present invention, there is provided an X-ray examination apparatus comprising:

an X-ray source for exposing an object to be examined to X-ray energy;
and an X-ray detector apparatus for generating image data representing an image of the object from an imaging unit,
a system according to any of the preceding claims and
a positioning system for positioning a carrier carrying the object.

According to a third aspect of the present invention, there is provided a method for positioning a carrier of an object within a field of view of an imaging apparatus,

the method comprising:

providing image data representing an image of the object,

determining object components position data representing positions of components of the object,

matching object components positions and anatomic model components positions based on object components position data and stored anatomic model data,

receiving input data representing a component of interest,

determining position shift data representing a direction and a distance by which the carrier is to be shifted, considering the input data, anatomic model data and object components position data,

shifting the carrier based on the position shift data.

Preferably, the object is a human body and wherein the components of the objects are at least one of: organs and bones of the human body.

Preferably, the carrier is a table.

Preferably, the image data represents a front view or rear view of the object.

Preferably, object position data represents a three-dimensional position of an object.

Preferably, the method further comprises displaying an image representing anatomic model components positions and object components positions.

Preferably, anatomic model components positions are average positions of the components within the object.

Preferably, the imaging apparatus is an X-ray apparatus.

Preferably, the imaging apparatus comprises a rotatable C-arm.

Preferably, the method is used in a cardiology examination or in a neurovascular examination.

According to a fourth aspect of the present invention, there is provided a Memory device comprising stored code means adapted to produce the steps of the methods when loaded into the memory of a computer.

The usually ill-posed 2D/3D matching problem, which occurs when fitting the model to a single x-ray projection, is simplified if the patient is lying with the back on a table top generating a rear view or front view of the patient's human body. This reduces the degrees of freedom for the matching of the positions of object components (organs, bones, rips etc) in an X-ray image with a stored anatomic model. Most important for the matching are the locations of bones and ribs because they are much more opaque in x-ray images than soft tissue.

The invention simplifies patient positioning and makes a C-arm system easier to use. Since full-body anatomic models are becoming available, the proposed system can be applied especially in cardiology but also in neurovascular examinations. If the anatomic models are detailed enough to include the locations of blood vessels (e.g., coronaries or cerebral vessels), the positioning can be done even on a vessel basis instead of an organ basis.

Other aspects, features and advantages of the present invention will become more fully apparent from the claims and the following detailed description of preferred embodiments thereof which is to be taken in conjunction with the appended drawings.

### BRIEF DESCRIPTION OF THE DRAWINGS

FIG. 1 shows the work flow of an embodiment of the invention.

FIG. 2 shows a C arm detection device.

FIG. 3 shows a C arm detection device with a table for a human body to be examined.

FIG. 4 shows an image with positions of organs and bones.

FIG. 5 shows an anatomic model with average positions of organs and bones.

FIG. 6 shows the anatomic model of FIG. 5 but with positions of organs and bones at positions that the organs and bones have in FIG. 4.

FIG. 7 shows components of an embodiment of the invention.

### DETAILED DESCRIPTION OF EMBODIMENTS

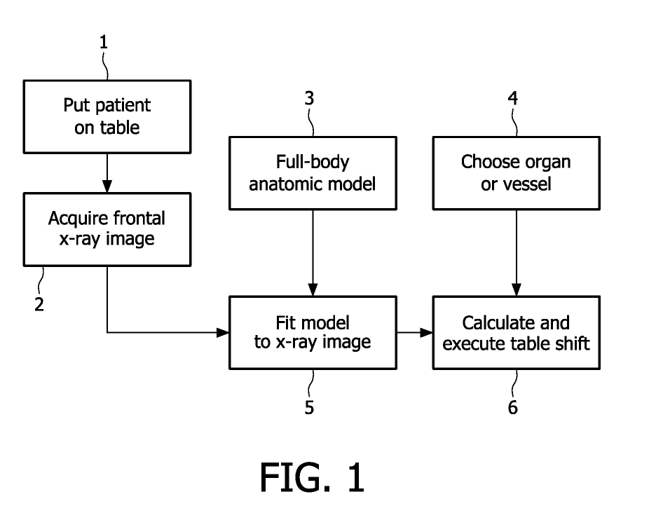

FIG. 1 shows a processing flow for an embodiment of the invention and further actions. The processing includes actions or input from the doctor (1, 2, 5), model knowledge (3), and automatic software modules (4, 6).

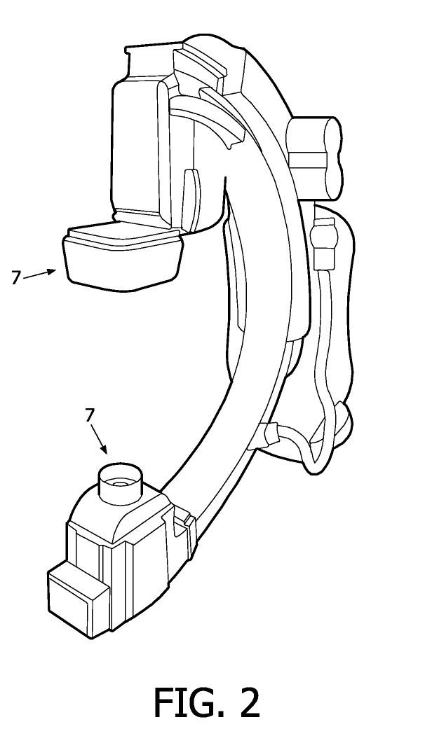

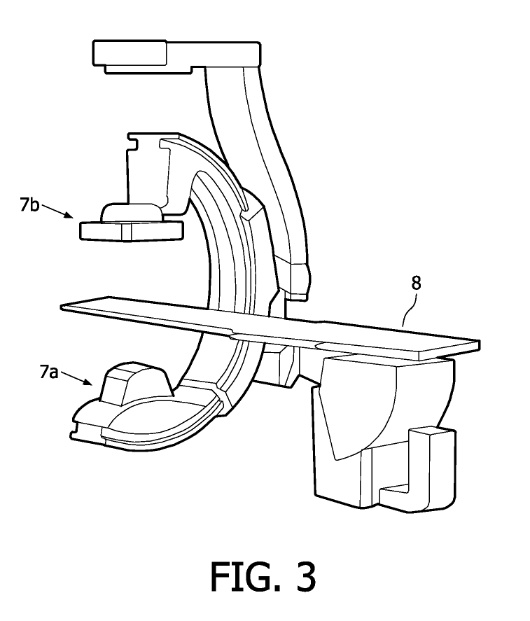

The workflow according to FIG. 1 is: An object as e.g. a human body is placed (1) on a table of an imaging unit 7 (e.g. X-ray imaging device in a C arm detection device) wherein the C-arm of an X-ray system (FIGS. 2, 3) is in a frontal (a. p.) position. Image data represented in an x-ray image (9) is acquired (2). Then a stored (16) anatomic model (9*) of a human body (10) is “deformed” or adapted (17) in a way that virtual x-ray projections of the model fit the actually acquired image (5). An image (9**) of the anatomic model—however with positions of organs and bones in this image according to the image data of the X-ray image—is presented to a doctor. Input (4, 23) from a doctor is received (5, 19), which input (23) chooses an object component (11**) of interest (e.g. by touching it on a touch screen or by input of a number etc), i.e. an organ to be examined, for example the heart 11** or a bone 12**. The system (24) calculates (6) position data (27) representing a shift that is required to move the given organ, known from the stored anatomic model, e.g. to the center of rotation of the C-arm (FIGS. 2, 3). The carrier of the object (e.g. a table 8) is automatically moved (21) according to the position shift data.

FIG. 2 shows a C arm detection device (7) for use with a system according to the invention.

FIG. 3 shows a C arm detection device with an X-ray source (7a), with an X-ray detector (7b) and with a table (8) for an object as e.g. a human body (not shown) to be examined.

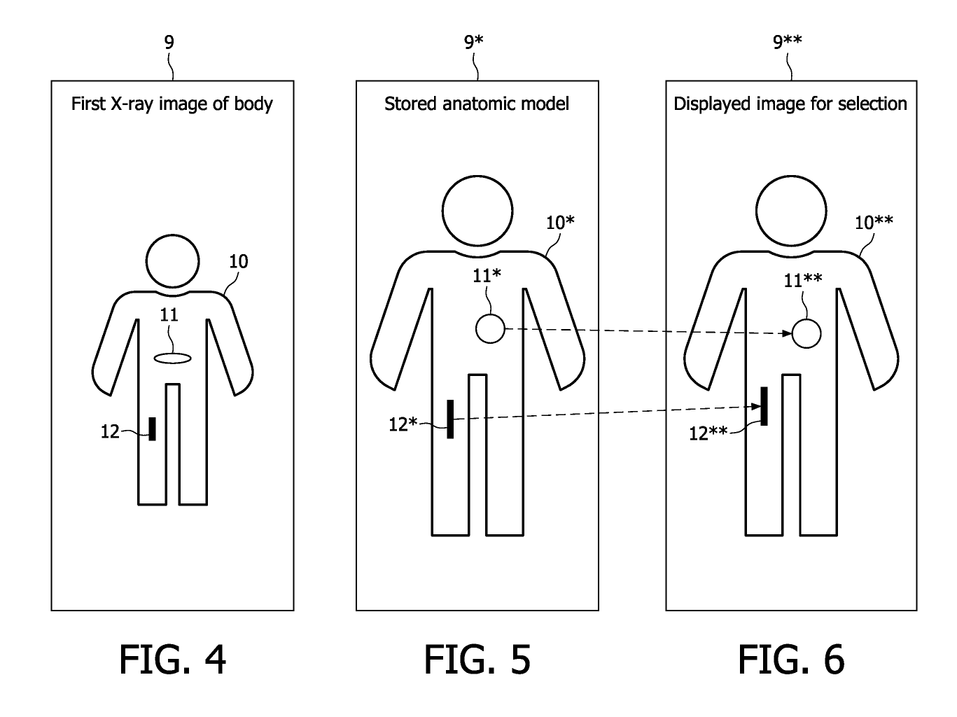

FIG. 4 very schematically shows an image based on image data (22) received from an imaging device (7b), the image showing a human (10) body with organs (11), bones (12) and rips etc.

FIG. 5 very schematically shows a stored anatomic model (9*) represented in stored (16) anatomic model data of an average human body (10*) with (stored) average positions of organs (11*) and bones (12*) and rips etc.

In FIG. 6 the organs (11**) and bones (12**) shown in the anatomic model in FIG. 5 are shifted to the positions that the real organs (11) and bones (12) of the body have in FIG. 4. The image (9**) in FIG. 6 can be displayed to a doctor who can input a selected component (11**) of interest (e.g. by a displayed number) and before the next X-ray will be taken the carrier with the body will be shifted by a positioning system (21) to a position in which position the selected component (11) of the body will be e.g. in a desired position resulting in a desired position (e.g. in the middle) within the next X-ray image to be taken then.

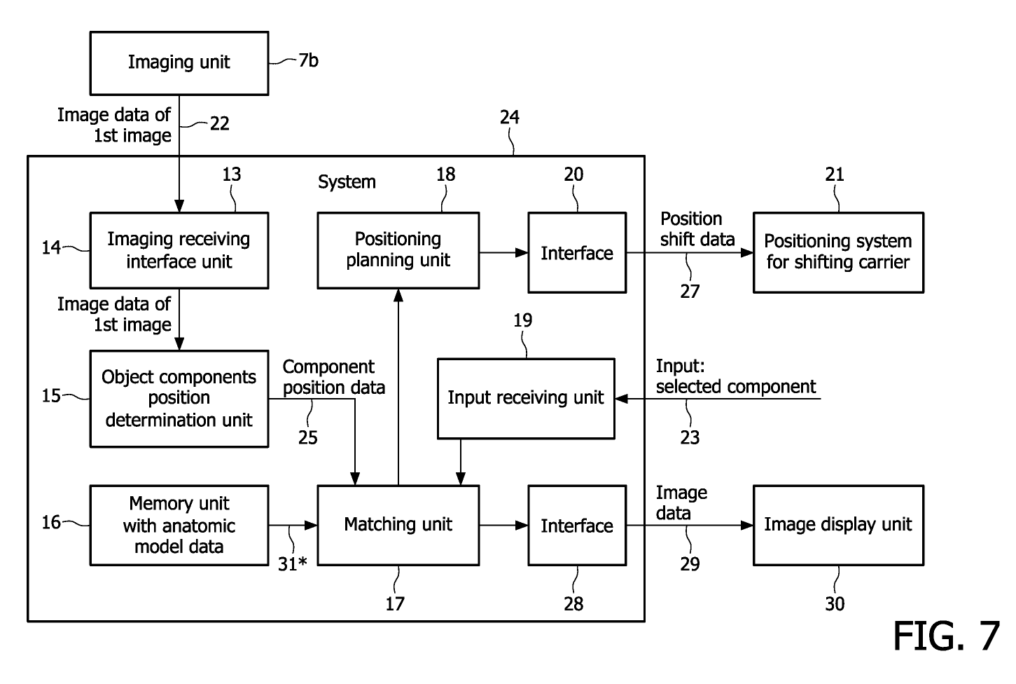

FIG. 7 shows components of an embodiment of the invention, i.e. a (controller) system (24) for positioning a carrier (9) of an object (10) within a field of view of an imaging unit (7b), the system comprising:

an imaging receiving interface unit (14) for receiving image data (22) representing an image (9) of the object (10) from an imaging unit (7b),

an object components position determination unit (15) for determining object components (11, 12) position data (25) representing positions of components (11, 12) of the object (10),

a memory (16) with anatomic model data stored therein, the anatomic model data representing anatomic model (9*) component positions of anatomic model components (11*, 12*) of an atomic model (10*) and image data for producing an image 9* as in FIG. 5 of the anatomic model,

a matching unit (17) designed to generate image data (29) representing an image (as e.g. the image 9* in FIG. 5 but with positions of the objects components or organs/bones 10, 11, 12 shifted in the image 9* to a position defined by the object positions of the objects 10, 11, 12 in the image 9 in FIG. 4, which image 9 of the object was generated before by an X-ray apparatus), which is to be sent via an interface (28) to an image display unit (30), based on object components (11, 12) position data (25) and anatomic model data (31*),

an input receiving unit (19) for receiving input data (23) representing a selected component (11) of interest,

an input receiving unit (19) for receiving input data (23) representing a selected component (11) of interest,

a positioning planning unit (18) for determining position shift data representing a direction and a distance by which the carrier (8) is to be shifted, considering the input data, anatomic model data and object components position data,

an interface (20) to a positioning system (21) for shifting the carrier (8) based on the position shift data (27).

What has been described above is what is presently considered to be a preferred embodiment of the present invention. However, as is apparent to the skilled reader, it is provided for illustrative purposes only and is in no way intended to that the present invention is restricted thereto. Rather, it is the intention that all variations and modifications be included which fall within the spirit and scope of the appended claims.

1. A system for positioning a carrier of an object within a field of view of an imaging unit,
the system comprising:
an image receiving interface unit for receiving image data, representing an image of the object, from the imaging unit,
an object components position determination unit for determining object components position data representing positions of components of the object,
an anatomic model data memory with anatomic model data stored therein, the anatomic model data representing anatomic model component positions of anatomic model components of an atomic model,
a matching unit designed to generate, based on object components position data and anatomic model data, image data representing an image, which image data is to be provided via an interface to an image display unit,
an input receiving unit for receiving input data representing a selected component of interest,
a positioning planning unit for determining position shift data representing a direction and a distance by which the carrier is to be shifted, depending on at least the input data,
an interface to a positioning system for shifting the carrier based on the position shift data.

2. The system according to claim 1,
wherein the object is a human body and wherein the components of the objects are at least one of: organs and bones of the human body.

3. The system according to claim 1, wherein the carrier is a table.

4. The system according to claim 1, wherein the image data represents only one image of a front view or rear view of the object.

5. The system according to claim 1, wherein the direction and the distance by which the carrier is to be shifted represent a three-dimensional vector.

6. The system according to claim 1, wherein it further comprises a display unit displaying an image representing anatomic model components positions and object components positions.

7. The system according to claim 1, wherein anatomic model components positions are average positions of the components within such objects.

8. The system according to claim 1 wherein the imaging apparatus is a cardio-vascular X-ray system or other X-ray system.

9. The system according to claim 1, wherein the imaging apparatus comprises a rotatable C-arm.

10. An X-ray examination apparatus comprising:
an X-ray source for exposing an object to be examined to X-ray energy;
and an X-ray detector apparatus for generating image data representing an image of the object from an imaging unit,
a system according to claim 1 and
a positioning system for positioning a carrier carrying the object.

11. A method for positioning a carrier of an object within a field of view of an imaging apparatus,
the method comprising:
receiving image data representing an image of the object,
determining object components position data representing positions of components of the object,
matching object components position data and stored anatomic model data to generate image data,
receiving input data representing a component of interest,
determining position shift data representing a direction and a distance by which the carrier is to be shifted,
causing the carrier to shift its position.

12. The method according to claim 11,
wherein the object is a human body and wherein the components of the objects are at least one of: organs and bones of the human body.

13. The method according to claim 11, wherein the carrier is a table.

14. The method according to claim 11, wherein the image data represents a front view or rear view of the object.

15. The method according to claim 11, wherein object position data represents a three-dimensional position of an object.

16. The method according to claim 11, wherein it further comprises displaying an image representing anatomic model components positions and object components positions.

17. The method according to claim 11, wherein anatomic model components positions are average positions of the components within the object.

18. The method according to claim 11, wherein the imaging apparatus is an X-ray apparatus.

19. The method according to claim 11, wherein the imaging apparatus comprises a rotatable C-arm.

20. The method according claim 11, wherein it is used in a cardiology examination or in a neurovascular examination.

21. A memory device comprising stored code means adapted to produce the steps of claim 11 when loaded into the memory of a computer.
